(19) United States
(12) Patent Application Publication
(10) Pub. No.: US 2024/0161923 A1
(43) Pub. Date: May 16, 2024

(54) VITAL SIGNS MONITORING AND COMMUNICATION

(71) Applicant: Welch Allyn, Inc., Skaneateles Falls, NY (US)

(72) Inventors: John A. Lane, Venice, FL (US); David E. Quinn, Moravia, NY (US); Alisa Robinson Salibra, Skaneateles, NY (US)

(21) Appl. No.: 18/497,306

(22) Filed: Oct. 30, 2023

Related U.S. Application Data
(60) Provisional application No. 63/383,859, filed on Nov. 15, 2022.

(51) Int. Cl. G16H 40/67; A61B 5/00; A61B 5/0205; G16H 80/00

(52) U.S. Cl. CPC G16H 40/67; A61B 5/0205; A61B 5/7282; A61B 5/7285; A61B 5/7405; A61B 5/7465; G16H 80/00

(57) ABSTRACT

A method of monitoring a subject includes measuring one or more vital sign values associated with the subject at a predetermined frequency. The method includes receiving a communication message from the subject, and tagging the communication message with a timestamp. The method includes transmitting the communication message and the one or more vital sign values measured proximate the timestamp to a computing device associated with a healthcare provider.

### BACKGROUND

Trained medical professionals regularly monitor the status of a patient under their care. Often, the status of the patient is monitored before, during, and after a medical visit or procedure. In some cases, the patient's visit to a healthcare facility may need to be extended so that the status of the patient can be regularly monitored by a medical profession. Often times, increasing the length of the patient visit may result in increased costs for the patient. In addition, due to shortages in medical professionals, medical facilities may also lack the resources to accommodate a patient for an extended length of time.

### SUMMARY

In general terms, the present disclosure relates to patient monitoring. In one possible configuration and by non-limiting example, a wearable device is used to measure vital signs and record patient communications. In another possible configuration and by non-limiting example, a wearable device adjusts a vital signs measurement rate based measured vital signs and/or recorded patient communications, and is preprogrammed to operate under different modes of operation based on a condition and/or a location of a patient. In another possible configuration and by non-limiting example, a wearable device pushes measured vital signs and/or recorded patient communications to a cloud server to facilitate remote patient monitoring. Various aspects are described in this disclosure, which include, but are not limited to, the following aspects.

One aspect relates to a device for monitoring a subject, the device comprising: one or more sensors; and a computing device including: one or more processors; and memory storing instructions, which when executed by the one or more processors cause the one or more processors to: use the one or more sensors to measure one or more vital sign values of the subject at a predetermined frequency; receive a communication message from the subject; tag the communication message with a timestamp; and transmit the communication message and the one or more vital sign values measured proximate the timestamp to a computing device associated with a healthcare provider.

Another aspect relates to a method of monitoring a subject, the method comprising: measuring one or more vital sign values of the subject at a predetermined frequency; receiving a communication message from the subject; tagging the communication message with a timestamp; and transmitting the communication message and the one or more vital sign values measured proximate the timestamp to a computing device associated with a healthcare provider.

Another aspect relates to a method of communicating with a healthcare provider using a monitoring device, the method comprising: measuring one or more vital sign values of a subject at regular time intervals; upon measuring the one or more vital sign values, transmitting the one or more vital sign values to a computing device associated with the healthcare provider for display; receiving a first communication message from the subject, wherein the first communication message is an audio message; determining whether the first communication message includes a preset keyword; upon determining that the first communication message does not include the preset keyword, tagging the first communication message with a first timestamp; transmitting the first communication message with the first timestamp and the one or more vital sign values measured proximate the timestamp to a computing device associated with the healthcare provider; receiving, at the monitoring device a second communication message from the computing device associated with the healthcare provider, wherein the second communication message includes an audio message responding to the first communication message; and interfacing with a speaker associated with the monitoring device and causing playback of the second communication message on the speaker.

The details of one or more techniques are set forth in the accompanying drawings and the description below. Other features, objects, and advantages of these techniques will be apparent from the description, drawings, and claims.

### BRIEF DESCRIPTION OF DRAWINGS

The following drawings are illustrative of particular examples of the present disclosure and therefore do not limit the scope of the present disclosure. The drawings are not to scale and are intended for use in conjunction with the explanations in the following detailed description. Examples of the present disclosure will hereinafter be described in conjunction with the appended drawings, wherein like numerals denote like elements.

FIG. 1 schematically illustrates an example of a patient monitoring system.

FIG. 2 illustrates an example of a schematic block diagram of a monitoring device included within the patient monitoring system of FIG. 1.

FIG. 3 illustrates an example of a configuration of a computing device of the monitoring device of FIG. 2.

FIG. 4 illustrates an example of a method of acquiring a vital sign data measurement using the patient monitoring system of FIG. 1.

FIG. 5 illustrates an example of a method of transmitting communications between a subject and a healthcare provider using the patient monitoring system of FIG. 1.

FIG. 6 illustrates an exemplary architecture of the computing device that can be used to implement aspects of the patient monitoring system of FIG. 1.

### DETAILED DESCRIPTION

Various examples will be described in detail with reference to the drawings, wherein like reference numerals represent like parts and assemblies throughout the several views. Reference to various examples does not limit the scope of the claims attached hereto. Additionally, any examples set forth in this specification are not intended to be limiting and merely set forth some of the many possible embodiments for the appended claims.

Patients visiting a healthcare facility for a routine check-up visit or to undergo a medical procedure may need to be monitored for one or more vital signs before, during and after the visit. Often times, the length of a patient's visit to the healthcare facility is extended so that the patient can be regularly monitored by medical professionals. However, extending the length the patient's visit may increase monetary costs for both the patient and the healthcare facility. In addition, with shortages in the number of medical professionals and hospital resources, medical facilities may have difficulty in allocating resources towards regularly checking up on the health status of the patient.

The disclosed patient monitoring system uses a monitoring device to remotely monitor the health status of the patient at regular intervals and communicate the health status to a healthcare provider. In addition, the disclosed patient monitoring system may facilitate the communication between the patient and the healthcare provider using a wearable monitoring device. For example, the patient may be able to record and send questions or comments regarding a symptom or health condition to the healthcare provider using the wearable monitoring device.

For example, the disclosed patient monitoring system may include a monitoring device that is configured to measure one or more vital signals associated with a patient. Vital signs that are monitored using the monitoring device may include heart rate, patient position, ambulation index, temperature of the patient, agitation index, ambient noise level and blood pressure. The monitoring device, upon measuring data related to one or more aspects of the health of the patient, may communicate the data to the healthcare provider. The healthcare provider may remotely monitor and review the received health data and respond to any abnormalities regarding the health status of the patient. For example, the healthcare provider may intervene upon identifying abnormalities regarding the health status of the patient by requesting that the patient visit the healthcare facility for follow-up testing and/or monitoring or by requesting the monitoring device to acquire and transmit additional data related to the health of the patient. The healthcare provider may respond in other ways as well.

In addition to the response from the healthcare provider when an abnormality is detected, the monitoring device may also automatically change the sampling rate of measuring health data on the subject. For example, upon detecting an abnormality in the measured health data, the monitoring device may automatically increase the frequency with which the monitoring device measures the vital signs of the subject.

In addition to sending data related to the health of the patient, the monitoring device may also be configured to allow the patient to communicate with the healthcare provider. For example, the monitoring device may include a microphone and speaker that may enable the patient to send messages or voice recordings to the healthcare provider.

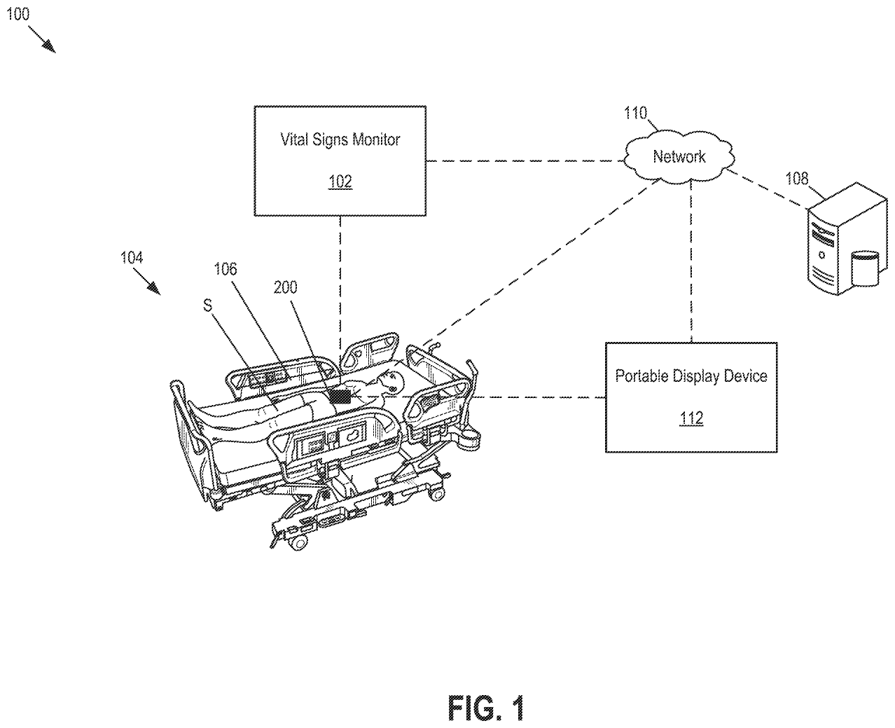

FIG. 1 schematically illustrates an example patient monitoring system 100. The patient monitoring system 100 includes a vital signs monitor 102 configured to measure and display one or more vital signs of a subject S located in a subject arrangement area 104. The vital signs are displayed on the vital signs monitor 102 numerically, in waveform, in tabular data, in plots of tabular data, and the like.

The subject S is a person, such as a patient, who is clinically treated by one or more medical professionals. As shown in the example of FIG. 1, the subject S is arranged in the subject arrangement area 104, which can be located inside a healthcare facility such as a hospital, medical clinic, doctor's office, and the like, or outside the healthcare facility, such as the subject's home. The subject arrangement area 104 can optionally include a support device 106, such as a bed, on which the subject S can lie, rest, or sleep. Other examples of the support device 106 include lifts, chairs, stretchers, and surgical tables. In some examples, the subject arrangement area 104 may not require a support device 106 at all.

The patient monitoring system 100 further includes a monitoring device 200 that is configured for attachment to the body of the subject S. The monitoring device 200 is a wireless patch that measures and monitors the vital signs of the subject S. In one example, the monitoring device 200 is attached to the chest of the subject S. On another example, the monitoring device 200 may be attached to another part of the subject's body. In one example, the monitoring device 200 is reusable after it has been used on the subject S. In another example, the monitoring device 200 is disposable after it has been used.

In one example, the monitoring device 200 is operable to wirelessly communicate with the vital signs monitor 102 via a data communication network 110. In another example, the monitoring device 200 is operable to directly communicate with the vital signs monitor 102 through a wireless connection. In a further example, the monitoring device 200 is operable to directly communicate with a portable display device 112 such as a smartphone, tablet computer, laptop, and the like through another wireless connection. In these examples, the vital signs of the subject S measured by the monitoring device 200 is displayed on the vital signs monitor 102 and/or on the portable display device 112.

In further examples, the monitoring device 200 is operable to wirelessly communicate with a data management system 108 via the data communication network 110. The data management system 108 operates to manage personal and/or medical information, such as health conditions and other information of the subject S. The data management system 108 can be operated by medical professionals or healthcare service providers.

The vital signs monitor 102 and portable display device 112 are also operable to communicate with the data management system 108 via the data communication network 110 to transmit data such as measured vital signs and other data associated with the subject S. In some examples, the vital signs monitor 102 and portable display device 112 are capable of directly communicating with the data management system 108, and/or with each other.

The data management system 108 may include one or more cloud-based computing systems. The data management system 108 operates to provide information that can be used to assist medical professionals to provide suitable healthcare to the subject S. In one example, the data management system 108 may provide a connection to electronic medical record or electronic health record servers. In an alternative example, the data management system 108 may itself be an electronic medical record server or an electronic health record server.

The data communication network 110 communicates data between computing devices, such as among the vital signs monitor 102, the portable display device 112, the monitoring device 200, and the data management system 108. Examples of the data communication network 110 include a local area network and a wide area network, such as the Internet. The data communication network 110 includes a wireless communication system, a wired communication system, or a combination of wireless and wired communication systems. Examples of wired communication systems can transmit data using electrical or optical signals in various possible configurations. Examples of wireless communication systems can transmit signals via electromagnetic waves such as in the form of optical signals or radio frequency (RF) signals. A wireless communication system can include a transmitter for transmitting optical or RF signals, and a receiver for receiving the optical or RF signals. Examples of wireless communication systems include Wi-Fi communication devices that utilize wireless routers or wireless access points, cellular communication devices that utilize one or more cellular base stations, Bluetooth, ANT, ZigBee, medical body area networks, personal communications service (PCS), wireless medical telemetry service (WMTS), and other wireless communications.

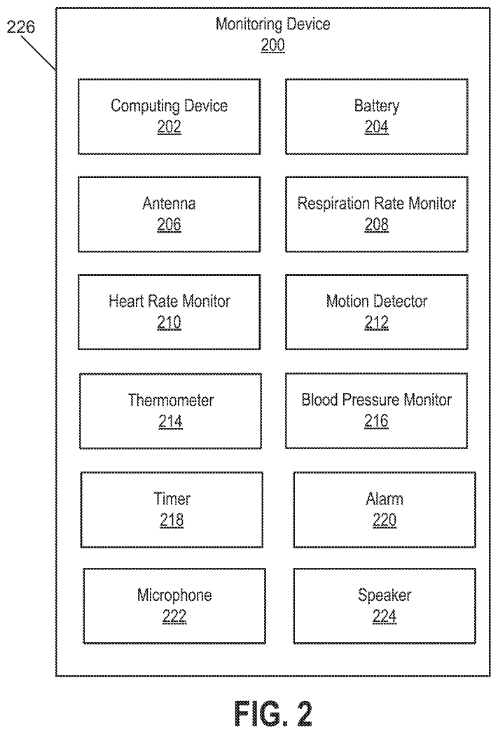

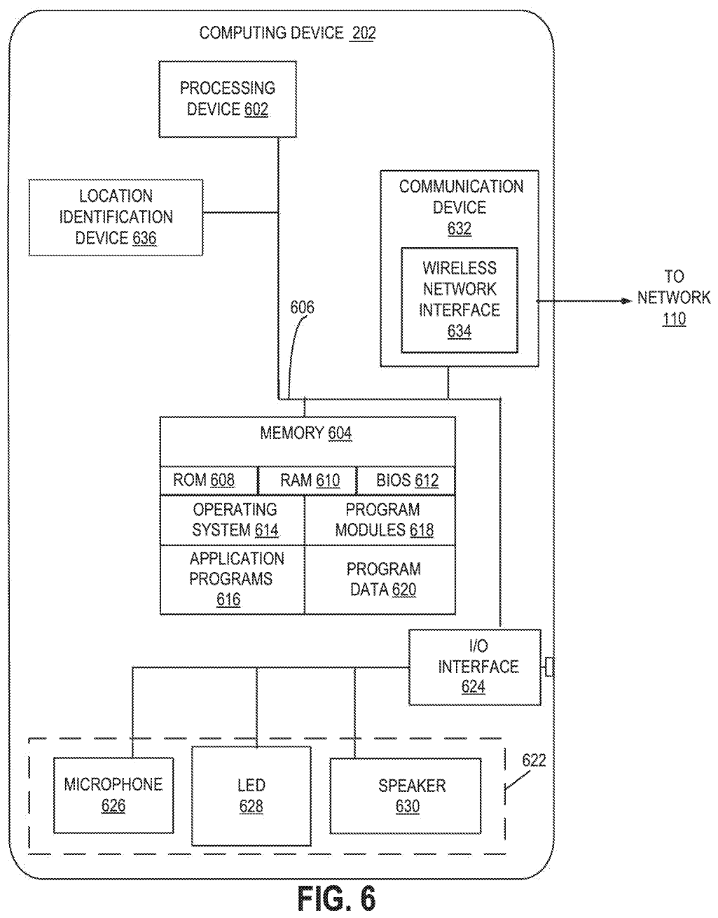

FIG. 2 is a schematic block diagram illustrating an example of the monitoring device 200 for detecting and measuring one or more vital signs of the subject S. The monitoring device 200 includes a battery 204, an antenna 206, and one or more sensors such as a respiration rate monitor 208, a heart rate monitor 210, a motion detector 212, a thermometer 214. The monitoring device 200 can further include a timer 218, an alarm 220, a microphone 222, and a speaker 224. Each component of the monitoring device 200 shown in FIG. 2 is operatively coupled to a computing device 202, which is described in more detail with reference to FIG. 6.

The components of the monitoring device 200, including the computing device 202, the battery 204, the antenna 206, the respiration rate monitor 208, the heart rate monitor 210, the motion detector 212, thermometer 214, the timer 218, the alarm 220, the microphone 222, and the speaker 224 are packaged together within a housing 226. The housing 226 is configured to environmentally protect these components when the monitoring device 200 is worn by the subject S. The housing 226 can be made from a durable, lightweight material such as plastic.

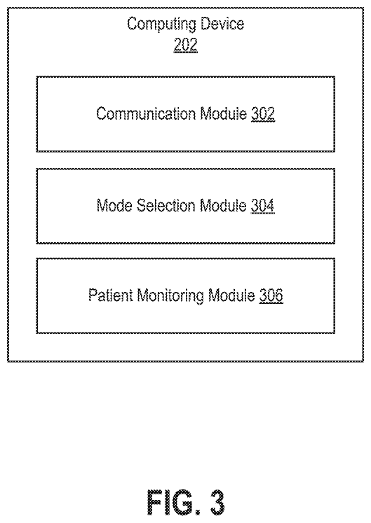

The computing device 202 includes at least a processor and a memory. In some examples, the computing device 202 may be a microprocessor. The computing device 202 controls operation of the antenna 206, the respiration rate monitor 208, the heart rate monitor 210, the motion detector 212, the thermometer 214, the blood pressure monitor 216, the timer 218, the alarm 220, the microphone 222, and the speaker 224 to acquire health data associated with the subject S and to enable communications between the subject S and the healthcare provider. The computing device 202 is described further with reference to FIGS. 3 and 6.

The battery 204 may be configured to provide power to the computing device 202, the antenna 206 the respiration rate monitor 208, the heart rate monitor 210, the motion detector 212, the thermometer 214, the blood pressure monitor 216, the timer 218, the alarm 220, the microphone 222, and the speaker 224. The battery 204 can be a replaceable and/or disposable battery. Alternatively, the battery 204 can be a rechargeable battery.

The antenna 206 may be configured to wirelessly transmit data acquired by the respiration rate monitor 208, the heart rate monitor 210, the motion detector 212, and the microphone 222, to the vital signs monitor 102, the portable display device 112, and/or the data management system 108. In some examples, the antenna 206 may be configured to wirelessly transmit the acquired data directly via Bluetooth or a Wi-Fi connection. In other examples, the antenna 206 may be configured to wirelessly transmit the acquired data via the data communication network 110.

The antenna 206 may also be configured to wirelessly receive data, instructions, and/or communications from the vital signs monitor 102, the portable display device 112, and/or the data management system 108. The antenna 206 may then interface with the computing device 202 to process the received data, instructions, and/or communications as described further in relation to FIG. 3.

The respiration rate monitor 208 may be configured to measure the respiration rate of the subject S. Respiration rate is the number of breaths a person takes per minute. The rate is usually measured when a person is at rest and involves counting the number of breaths per minute. The monitoring device may also monitor the depth, variation and escalation associated with the respiration rate of the patient.

In some examples, the respiration rate monitor 208 may be configured to include a first transceiver and a second transceiver. For example, the first and second transceivers may include piezo crystal oscillators that are operable to transmit pulses for measuring respiration rate. The pulses may measure a distance between a chest wall and a lung cavity of the subject S, which changes when the subject S breathes due to the lung cavities filling and expanding with air. In one example, the pulses are ultrasonic. In this example, acoustic impedance (e.g., a measure of the opposition to the acoustic flow resulting from the pulses) may be detected from reflected pulses, and is used to measure the respiration rate of the subject S. Other methods of measuring the respiration rate may also be possible. The respiration rate monitor 208 is further described in U.S. application. Ser. No. 16/84587, which is hereby incorporated by reference.

The heart rate monitor 210 may be configured to measure the heart rate of the subject. Heart rate is the frequency of the heartbeat measured by the number of contractions of the heart per minute. The heart rate can vary based on the subject's age, genetics, physical fitness, stress or psychological status, diet, drugs, hormonal status, environment, disease or illness and other factors. In addition to measuring the heart rate, the heart rate monitor 210 may also be configured to identify irregularities, including atrial fibrillation conditions and measure heart rhythm data using electrocardiography.

The heart rate monitor 210 may be configured to measure heart rate by using electrical detection to track the heart rate of the subject. For example, the heart rate monitor may include electrodes that detect electrical signals produced by the heart. In other examples, the heart rate monitor 210 may also detect heart rate through photoplethysmography (PPG), or the process of using light to measure blood flow. In such an example, the heart rate monitor 210 may include small Light Emitting Diodes (LEDs) that shine green light onto the skin on the subject's wrist when the monitoring device 200 is worn around the wrist of the patient and a sensor to measure the light refracted after interacting with the blood of the subject. Other ways of measuring the heart rate of the subject are also possible.

The motion detector 212 is configured to measure motion of the subject. The motion of the subject may be monitored by the healthcare provider in order to determine an ambulation index and/or agitation index of the person. An ambulation index measures the rate at which the subject is moving in and out of bed. An agitation index measures whether the patient is shifting constantly above a threshold level. The ambulation index and the agitation index allow the healthcare provider to determine the comfort level and mobility level of the subject. The motion detector 212 may be configured to include one or more motion sensors, accelerometers, and/or a pedometer to measure the movement of the subject.

The thermometer 214 may be configured to measure the temperature of the subject. The thermometer 214 may be an electronic thermometer that is configured to apply a voltage across a metal probe and measuring the resistance. Other types of thermometers can also be used.

The blood pressure monitor 216 may be configured to measure the blood pressure of the subject. The monitoring device 200 may include an inflatable cuff that may be used to collapse and release the artery under the cuff in a controlled manner to measure the blood pressure of the subject S. The blood pressure monitor 216 can be configured to measure the blood pressure of the subject in other ways as well.

The timer 218 alternates the operation of the monitoring device 200 between a sleep mode and an active mode. In the sleep mode, energy from the battery 204 is conserved. The timer 218 automatically wakes up the monitoring device 200 from the sleep mode to operate under the active mode at predetermined time intervals. The time intervals may be determined by the computing device 202 and is described in greater detail in relation to FIG. 3. By alternating between the sleep and active modes, the monitoring device 200 preserves the life of the battery 204.

The alarm 220 is configured to generate an alarm signal when vital signs data acquired by any of the sensors of the monitoring device 200 such as the respiration rate monitor 208, the heart rate monitor 210, the motion detector 212, the thermometer 214, and/or the blood pressure monitor 216 of the monitoring device 200 is below a minimum threshold, is above a maximum threshold, or is indicative of a trend for an impending adverse event. An algorithm can be used to analyze the vital signs data acquired by the one or more sensors of the monitoring device 200 to generate the alarm signal. In one example, the alarm 220 is an audible alarm such as a beeping noise. In another example, the alarm 220 is a visual alarm such as a blinking light.

In an example, the monitoring device 200 may include a microphone 222 that detects whether the subject S is talking. Talking may affect some vital signs associated with the subject S because a person is not able to talk and breathe at the same time. Thus, the microphone 222 can reduce false alarms. The microphone 222 is operatively coupled to the computing device 202. In one example, when the microphone 222 detects that the subject S is talking, the microphone 222 causes the monitoring device 200 to operate in the sleep mode, such that the respiration rate monitor 208 does not transmit the pulses used to measure and/or monitor the respiration rate of the subject S. Thus, the microphone preserves the life of the battery 204 when the subject S is talking.

In further examples, the microphone 222 is configured to distinguish between regular talking, and other audible sounds made by the subject S such as snoring while sleeping or moaning when in pain. By distinguishing these audible sounds, the monitoring device 200 remains in the active mode when the subject S is sleeping or in pain.

The microphone 222 may also be used to measure ambient noises in the environment of the subject. The ambient noise measurement may be used by the monitoring device to evaluate whether the environment of the subject S is too loud or noise.

In addition to using the microphone 222 to detect that the subject is talking in order to increase the accuracy of the data collected by the respiration rate monitor 208, the microphone 222 may also be operatively connected to the computing device, which may be locally or remotely integrated with voice assistant technology. The subject S may be able to enable the voice assistant by uttering a specific key word. The voice assistant may thereafter actively listen to the words uttered by the subject through the microphone 222 and may offer responses or help with a task as applicable.

The microphone 222 may also be used by the subject S to record comments and/or questions to send to the healthcare provider. The recorded comments and/or questions can be sent to the healthcare provider as a voice recording, or can be transcribed to text automatically via a voice to text software application and sent to the healthcare provider thereafter.

In an example, the monitoring device 200 may also include a speaker 224 that may be used to playback voice recordings from a healthcare provider, playback responses from the voice assistant application, or be used for playback of any other recordings or sounds including reminders, alarms, alerts, and notifications.

FIG. 3 illustrates an example configuration of the computing device 202 of the monitoring device 200 of FIG. 1. In some examples, the computing device 202 may be configured to include a communication module 302, a mode selection module 304, and patient monitoring module 306. In other examples, the computing device 202 may be configured to include more or fewer number of modules.

The communication module 302 may be configured to interface with the antenna 206, the microphone 222, and the speaker 224 to receive and transmit communications between the monitoring device 200, and one or more of: the vital signs monitor 102, portable display device 112 and the data management system 108. In one example, the communication module 302 may receive communications from the subject S via the microphone 222 for transmission to a health care provider. Upon receiving the voice communication from the microphone 222, the communication module 302 may interface with the antenna 206 to send the communication to the data management system 108, where a healthcare provider may receive, review, and respond to the communication. In some cases, the communication module 302 may transmit the voice communication as an audio file. In other cases, the communication module 302 may use a voice to text application to translate the voice communication to text before transmitting the communication as a text communication.

The voice communication from the subject S, may include questions or provide notes regarding a symptom or health condition. In some cases, the communication module 302 may tag a time code to the voice recording such that the healthcare provider receiving the communication may correlate the subject S's comments or questions to a particular recorded vital sign data event.

For example, when the patient experiences symptoms such as dizziness, the subject S may record a voice memo, such as “I just felt dizzy for the last minute or so” via the microphone 222. The communication module 302 may tag the time at which the voice memo is recorded and transmit the voice memo along with the time to the data management system 108 such that a healthcare provider may listen to the voice memo and use the tagged time to look at data from the heart rate monitor 210 and respiration rate monitor 208 at or around the tagged time. The tagged time information can be used by the data management system 108 to automatically correlate the vital signs data to the voice memo without requiring the healthcare provider to manually search and find the vital signs data that corresponds to the tagged time.

In addition, the communication module 302 may interface with a voice assistant application similar to Google Alexa or Apple Siri to respond to automatically to enquiries from the subject S when the voice memo includes a specific key word. In some examples, when the subject S uses a keyword, such as “Hey Monitor,” followed by a question regarding their health, the communication module 302 may receive the communication from the microphone 222 and interface with the voice assistant application to provide a response to the subject's enquiry. The communication module 302 may send the response from the voice assistant application to the speaker 224 for playback on the monitoring device 200.

In some examples, the voice assistant application may be included locally within the computing device 202. In other example, the voice assistant application may be configured as an application within the vital signs monitor 102 or portable display device 112. The communication module 302 may interface with the voice assistant application configured within the vital signs monitor 102 or the portable display device 112 via wireless communications such as a Bluetooth connection.

In another example, the communication module 302 may receive communication from a healthcare provider via the antenna 206. For example, the voice or text communication from a healthcare provider regarding a question from the subject S, or in response to any collected vital sign data regarding the health of the subject S. For example, a healthcare provider may, using the vital signs monitor 102, the portable display device 112 or the data management system 108, record a voice or text message and transmit the message to the monitoring device 200. The antenna 206 may receive the transmitted message and send the message to the communication module 302 for further processing. In some examples, the communication module 302 may determine that the message is in an audio format and interface with the speaker 224 to enable playback of the received message. In other examples, the communication module 302 may determine that the message is in a text format and use a text to voice application to convert the received message to a voice format before interfacing with the speaker 224 and enabling playback of the received message.

In yet another example, the communication module 302 may receive communications from one or more of: the vital signs monitor 102, portable display device 112 and the data management system 108 via the antenna 206. For example, the communication module 302 may receive communications related to device settings associated with one or more components of the monitoring device 200. The communications may be transmitted based on inputs from the subject S or the healthcare provider via the vital signs monitor 102, portable display device 112 and the data management system 108.

In some examples, upon receiving the communication that includes device setting information, the communication module 302 may interface with and send the received communication to the patient monitoring module 306, for the patient monitoring module to adjust one or more settings associated with one or more components of the monitoring device 200, including the battery 204, the antenna 206, the respiration rate monitor 208, the heart rate monitor 210, the motion detector 212, the thermometer 214, the blood pressure monitor 216, the timer 218, the alarm 220, the microphone 222 and the speaker 224. In other examples, the communication module 302 may directly control the operation of the one or more components of the monitoring device 200 by adjusting one or more device settings associated with the one or more components.

Device settings associated with one or more components of the monitoring device 200 may include any setting that controls the operation of the component itself. For example, the device settings may control the frequency with which the respiration rate monitor 208, the heart rate monitor 210, the motion detector 212, or the thermometer 214 collects and records vital sign data associated with the subject S. Other examples of device settings may include adjusting the volume of the speaker 224, enabling the voice to text application associated with the communication module 302, changing settings related to the battery 204 to conserve energy, and additional types of device settings are contemplated. The device settings may include other options that control operation of the one or more components of the monitoring device 200 based on the preferences or requirements of the healthcare provider or the subject S to adequately capture any necessary vital sign data related to the health of the subject S.

The mode selection module 304 is configured to monitor vital sign data collected by the one or more components of the monitoring device 200 and determine whether to change the mode of operation of one or more components. For example, the one or more components of the monitoring device 200 may typically operate under one of a plurality of pre-formatted modes. Some example modes of operations include a pre-surgical mode, a medical-surgical ward mode, a post-surgical mode, and a home mode. Other types of modes of operations are also possible and can be customized based on the needs of the subject S and the healthcare provider.

In some examples, the pre-surgical mode may correlate with collecting vital sign data on the subject S prior to the subject S undergoing a medical procedure that may include surgery. The medical-surgical ward mode may correlate with collecting vital sign data on the subject S immediately before, during and immediately after the medical procedure. The post-surgical mode may correlate with collecting vital sign data on the subject S after the medical procedure, when the subject S is in recovery within the healthcare facility. The home mode may correlate with collecting vital sign data when the subject S is located in a location outside the healthcare facility, including the subject's home, after the subject S has been released from the healthcare facility following the medical procedure.

Each pre-formatted mode of operation may correlate to the frequency with which the respiration rate monitor 208, the heart rate monitor 210, the motion detector 212 and the thermometer 214 collect vital sign data on the subject S. For example, typically, one or more of the respiration rate monitor 208, the heart rate monitor 210, the motion detector 212 and the thermometer 214 may be prompted to collect vital sign data on the subject S most frequently in the medical-surgical ward mode and less frequently in the home mode.

In one example, one or more of the respiration rate monitor 208, the heart rate monitor 210, the motion detector 212 and the thermometer 214 may be prompted to collect vital sign data on the subject S every 10 minutes when the components are in the medical-surgical ward mode, but may only be prompted to collected on the subject S every 60 minutes when the components are in the home mode. The vital sign data collection frequency value associated with the each of the pre-formatted modes of operation is adjustable and may be adjusted by the healthcare provider using the vital signs monitor 102, the portable display device 112 or the data management system 108.

While the pre-formatted mode of operation of the components of the monitoring device 200 may be adjusted by the healthcare provider using the vital signs monitor 102, the portable display device 112 or the data management system 108, the mode selection module 304 may provide an additional method of automatically determining when a change in mode of operation of the one or more components is needed. For example, the mode selection module 304 may be configured to continuously analyze the vital sign data collected by the one or more components of the monitoring device 200 for any unexpected or abnormal values and, in response, automatically determine that an increase or decrease in the frequency of vital sign data collection is necessary to fully understand the collected values.

For example, the mode selection module 304 may continuously monitor the vital sign data collected from the one or more components of the monitoring device 200. The mode selection module 304 may also analyze whether the collected vital sign data is within an acceptable range of values expected for the subject S. Upon determining that the collected vital sign data is outside the acceptable range of values provided by the healthcare provider, the mode selection module 304 may deem the value to be unexpected or abnormal. When the mode selection module 304 identifies an unexpected or abnormal value in the collected vital sign data a threshold number of times over a threshold time period, the mode selection module 304 may automatically determine that the vital sign data may need to be collected more frequently. In some examples, the threshold number of times may be once. In other examples, the threshold number of times may be higher. In some examples, the threshold time period may be within an hour. In other examples, the threshold time period may be higher or lower.

Upon determining any changes to the mode of operation of the one or more components, the mode selection module 304 may be configured to send communication to the patient monitoring module 306 to execute the changes to the device settings of the one or more components of the monitoring device 200 to match the determined changes to the mode of operations.

For example, the mode selection module 304 may continuously monitor the vital sign data collected by the respiration rate monitor 208. Upon determining that the respiration rate is higher than expected for the subject S twice in the last hour, the mode selection module 304 may determine that the respiration rate monitor 208 may need to collect additional vital sign data. The mode selection module may thus make the determination that frequency with which the respiration rate monitor 208 collects respiration rate on the subject S need to be increased from once every 20 minutes to once every 10 minutes. Upon making the determination to increase the frequency with which to collect respiration rate data, the mode selection module 304 may send communication to the patient monitoring module 306 to execute the change. Subsequently, the patient monitoring module 306 may interface with the respiration rate monitor 208 to adjust the vital sign data collection frequency of the respiration rate monitor 208.

The patient monitoring module 306 may be configured to control the operations of the one or more components of the monitoring device 200 based on communications received from the communication module 302 and the mode selection module 304. In some examples, the patient monitoring module 306 may be configured to receive communications regarding controlling the operation of the one or more components of the monitoring device 200 from the communication module 302 and/or the mode selection module 304. In one example, upon receiving the communication with device setting information from the communication module 302, the patient monitoring module 306 may interface with the one or more components of the monitoring device 200 to adjust the device setting information associated with each of the one or more components related to the received communication.

For example, the communication module 302 may receive a communication from portable display device 112 to increase the frequency with which the respiration rate monitor 208 measures the respiration rate of the subject S by measuring the respiration rate of the subject every 10 minutes instead of every 30 minutes. Such a communication may be received by the antenna 206, and be sent to the communication module 302. The communication module 302 may then send the communication to the patient monitoring module 306 for the patient monitoring module 306 to interface with the respiration rate monitor 208 and adjust the settings of the respiration rate monitor 208 to measure respiration rate of the subject S every 10 minutes.

In another example, upon receiving communication from the mode selection module 304, the patient monitoring module 306 may interface with the one or more components of the monitoring device 200 to adjust the device setting information associated with each of the one or more components of the monitoring device 200 related to the communication from the mode selection module 304. For example, the patient monitoring module 306 may receive communication from the mode selection module 304 that the heart rate monitor 210 needs to be set in home mode. In such a case, the patient monitoring module 306 may interface with the heart rate monitor 210 to adjust the settings of the heart rate monitor 210 to a home mode, which in some examples includes decreasing the frequency with which the heart rate monitor 210 measures and records the heart rate of the subject S.

In another example, the patient monitoring module 306 may automatically adjust the mode based on a detected location of the monitoring device 200. For example, the monitoring device 200 may detect a location using a global positioning system (GPS) device integrated to the monitoring device 200 (not shown in the figures) or by detecting a connection or lack thereof to a recognized wireless network. For example, when the monitoring device 200 identifies a wireless network that the monitoring device 200 recognizes as the home network of the subject S, the monitoring device 200 may automatically change the monitoring device 200 to operate in a home mode.

The patient monitoring module 306 may also be configured to manage the vital sign data measured by the one or more components of the monitoring device, including the battery 204, the antenna 206, the respiration rate monitor 208, the heart rate monitor 210, the motion detector 212, the thermometer 214, the timer 218, the microphone 222 and the speaker 224. For example, the patient monitoring module 306 may receive collected vital sign data from one or more components of the monitoring device 200, including the respiration rate monitor 208, the heart rate monitor 210, the motion detector 212, and the thermometer 214.

Upon receiving any collected vital sign data, the patient monitoring module 306 may communicate the collected vital sign data to the mode selection module 304 for further processing and analysis. The patient monitoring module 306 may also interface with communication module 302 to transmit the received vital sign data to one or more of: the vital signs monitor 102, the portable display device 112, and/or the data management system 108.

In some examples, the patient monitoring module 306 may transmit the received data for display on the vital signs monitor 102 or portable display device 112. In some examples, the patient monitoring module 306 may transmit the collected vital sign data to the vital signs monitor 102 or portable display device 112 periodically at regular intervals. In other examples, the patient monitoring module 306 may transmit the collected vital sign data upon receiving the collected vital sign data from the one or more components of the monitoring device 200.

The patient monitoring module 306 may also interface with communication module 302 to transmit the received vital sign data to the data management system 108 via the antenna 206. The data management system 108 may include a cloud computing device where the collected vital sign data received from the monitoring device 200 may be uploaded, ready to be accessed by a healthcare provider.

In some examples, the patient monitoring module 306 may transmit the collected vital sign data to the data management system 108 periodically at regular intervals. In other examples, the patient monitoring module 306 may continuously transmit the collected vital sign data to the data management system 108 upon receiving the collected vital sign data from the one or more components of the monitoring device 200.

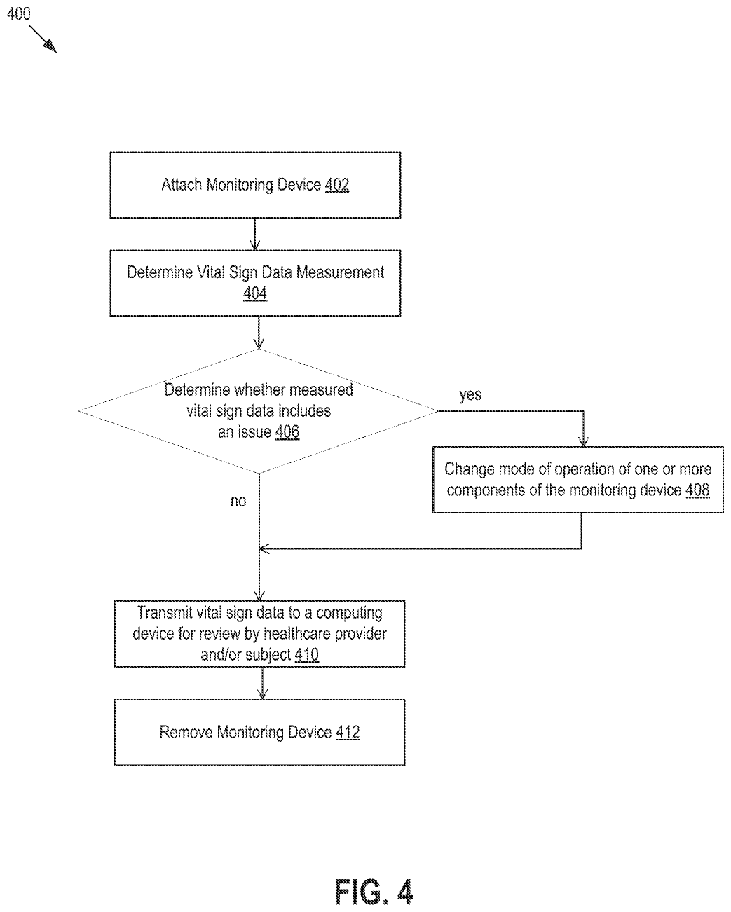

FIG. 4 illustrates an example of a method 400 of acquiring a vital sign data measurement. The method 400 includes placing the monitoring device 200 on the subject S (operation 402), determining a vital sign data measurement (operation 404), and removing the monitoring device from the subject (operation 412).

In operation 402, the monitoring device 200 is a wearable device. For example, the monitoring device 200 can be attached to the chest wall of the subject S such as by using a disposable adhesive. Alternatively, the monitoring device 200 may be worn by the subject S such as on a collar, wristband, or other type of accessory worn by the subject S, or by being placed inside a pocket of an article of clothing worn by the subject S. In such examples, the monitoring device 200 is not attached directly to the patient. The monitoring device 200 is placed on the subject S in operation 402 without requiring a breathing tube or nasal cannula, or any type of tether such as an electrode attached to an end of a cable. This allows the subject S to be mobile such that the subject S is able to freely walk about a healthcare facility or their own home with the monitoring device 200 attached to or otherwise worn by the subject S. The monitoring device 200 may be worn by the subject S for an extended period of time such as for 14 days or more.

In operation 404, determining a vital sign data measurement may include determining a respiration rate of the subject S, a heart rate of the subject S, an ambulation index of the subject S, an agitation index S, temperature of the subject S, ambient noise in an environment of the subject S and/or blood pressure of the subject S. Additionally, determining a vital sign data measurement can include determining the quality and depth of respiration, the uniformity of breath, irregularity of the heartbeat of the subject S, atrial fibrillation, and the position of the subject S. Other types of vital sign data may also be determined using the monitoring device 200 in operation 404.

The method 400 further includes an operation 406 of determining whether the measured vital sign data includes a sensed issue, such as an irregular, an unexpected, or an abnormal value(s) for one or more vital signs. In some examples operation 406 includes determining whether the measured vital signal data is outside an expected range of values for the subject S. Upon determining that the measured vital sign data may include an irregular, an unexpected, or an abnormal value (i.e., “Yes” in operation 406), the method 400 can proceed to an operation 408 of changing a mode of operation of one or more components of the monitoring device 200. In some examples a single irregular, unexpected or abnormal value in the measured vital sign data may result in the determination of a sensed issue and trigger the operation 406 to conclude and operation 408 to begin. In other examples, a threshold number of irregular, unexpected or abnormal values over a threshold time period may need to be identified for a determination of a sensed issue and for operation 406 to conclude and operation 408 to begin.

Upon determining that the measured vital sign data does not include irregular, unexpected or abnormal values to cause a determination of a sensed issue (i.e., “No” in operation 406), the operation 406 may conclude and trigger an operation 410 of transmitting the vital sign data to a computing device for review by the healthcare provider and/or subject S.

Changing the mode of operation of one or more components of the monitoring device 200 in operation 408 can include, when an issue is sensed in operation 406, the monitoring device 200 automatically determining that an increase or decrease in the frequency of vital sign data measurements is necessary to fully understand the collected data values. As a result, the patient monitoring module 306, in operation 408, may interface with one or more components of the monitoring device 200 in order to change the device settings such that the one or more components makes vital sign data measurements at an increased or decreased frequency.

In operation 410, transmitting vital sign data to a computing device for review by a healthcare provider and/or subject S can include, for example, transmitting the measured vital sign data to the vital signs monitor 102, the portable display device 112 and/or the data management system 108 for the healthcare provider or the subject S to view and analyze the collected data.

In operation 412, the monitoring device 200 is removed from the subject S in some examples by detaching the disposable adhesive from the chest wall of the subject. In further examples, the monitoring device 200 can be removed from the subject S such as by removing the collar, accessory, or article of clothing worn by the subject S. In some examples, the monitoring device 200 is a disposable device that is discarded after use. In other example embodiments, the monitoring device 200 is reusable. In further examples, certain components of the monitoring device 200 may be disposable while other components are reusable.

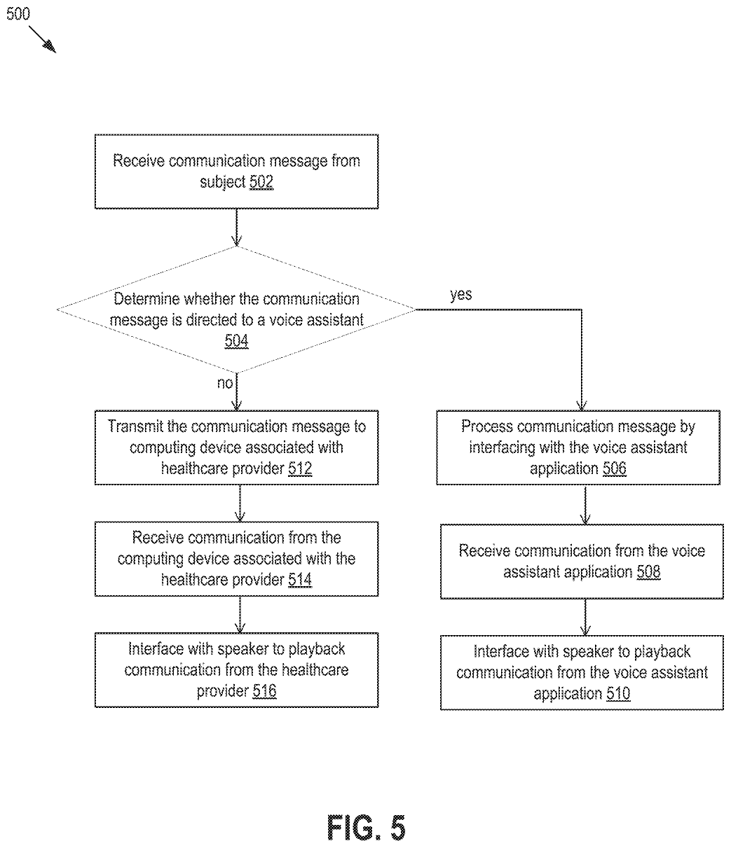

FIG. 5 illustrates a method 500 of transmitting communications between subject S and healthcare provider. The method 500 includes receiving a communication message from the subject (operation 502), determining whether communication message is directed to a voice assistant (operation 504), if directed to a voice assistant (i.e., “Yes” in operation 504), processing it accordingly (operations 506-510), if not directed to a voice assistant (i.e., “No” in operation 504), transmitting the communication message to healthcare provider (operation 512), receiving communication back from the healthcare provider (operation 514) and playing the communication from the healthcare provider over the speaker (operation 516).

In operation 502, the monitoring device 200 may receive a communication message from the subject S. For example, the subject S may record a communication message using the microphone 222 of the monitoring device 200. In some examples, the communication message may be a question or comment for the healthcare provider. In other examples, the communication message may be an inquiry directed at a voice assistant. The received communication message may be received via the microphone 222 and may be sent to the computing device 202 for processing by the communication module 302.

In operation 504, the communication module 302 determines whether the communication message is directed to a voice assistant, or not. For example, the communication module 302 may analyze the communication message received in operation 502 for a preset keyword such as, “Hey Monitor.” Upon determining that the communication message includes a preset keyword or key phrase, the communication module 302 may determine that the communication message is an inquiry directed to a voice assistant application and proceed with operations 506-510. Alternatively, upon determining that the communication message does not include a preset keyword or key phrase, the communication module 302 may determine that the communication message may include a question or comment for consideration by the healthcare provider and may proceed with operations 512-516 instead.

In operation 506, the communication module 302 may analyze the communication message and interface with the voice assistant application. In some examples, the computing device 202 may be configured to include a voice assistant application. In other cases, the computing device 202 may be configured to interface with a voice assistant application that is located externally of the monitoring device 200 via the data communication network 110.

In operation 508, the communication module 302 may receive a response communication back from the voice assistant application. For example, the voice assistant application may receive the communication message from operation 506 and process the inquiry included within the communication message. The voice assistant application may then respond to the inquiry by transmitting a response communication, such as an audio message, back to the communication module 302.

In operation 510, the communication module 302 may interface with the speaker 224 of the monitoring device 200 to initiate playback of the response communication received from the voice assistant application.

In operation 512, the communication module 302 may transmit the communication message received from the subject S in operation 502 to a computing device associated with a healthcare provider. When the communication message is analyzed and deemed not to include a preset keyword in operation 504, the communication message is deemed to likely include a question or comment for the healthcare provider. As a result, the communication message is transmitted by the monitoring device 200 to one or more of the vital signs monitor 102, the portable display device 112, and the data management system 108 via the antenna 206 for review by the healthcare provider.

In some examples, the communication message, which may include a voice communication message, may be transmitted as an audio file. In other examples, the communication module 302 may interface with a voice to text application to convert the communication message to text and transmit the text as a text message, email, instant message to a computing device associated with the healthcare provider.

In some examples, the communication module 302 may tag a time stamp of when the communication message was received from the subject S in operation 502 before transmitting the communication message. In addition to the time stamp tag, the communication module 302 may also include vital sign data measurements within a threshold amount of time surrounding the time stamp. For example, the communication module 302 may include a timestamp and vital sign data measurements for 10 minutes surrounding the time when the subject S initiated the communication when transmitting the communication message to the computing device associated with the healthcare provider.

In operation 514, the communication module 302 may receive a response communication back from the computing device associated with the healthcare provider as a response to the transmitted communication message from operation 512. For example, the response communication from the computing device associated with the healthcare provider may be an audio message or a text message. In some examples, when the response communication is a text message, the text message may be displayed on the vital signs monitor 102 or the portable display device 112 for review from the subject S. In other examples, when the response communication is a text message, the communication module 302 may interface with a text to voice application to convert the text message to an audio file.

In operation 516, the communication module 302 may interface with the speaker 224 of the monitoring device 200 to initiate playback of the response communication received from the computing device of the healthcare provider.

FIG. 6 illustrates an exemplary architecture of the computing device 202 that can be used to implement aspects of the present disclosure, including the monitoring device 200. The computing device 202 is used to execute the various functions, methods, or operations described herein. The monitoring device 200 can include all or some of the elements described with reference to FIG. 6, with or without additional elements.

The computing device 202 includes at least one processing device 602, such as a central processing unit (CPU). The computing device 202 also includes a memory 604, and a bus 606 that couples various system components including the memory 604 to the at least one processing device 602. The bus 606 can be of a variety of bus architectures.

The memory 604 may include read only memory 608 and random access memory 610. A basic input/output system 612 containing the basic routines that act to transfer information within the computing device 202 is typically stored in the read only memory 608. A number of program modules can be stored in the memory 604, including an operating system 614, one or more application programs 616, other program modules 618, and program data 620.

The computing device 202 can include various input and peripheral devices 622 such as a microphone 626, Light-Emitting Diodes (LED) 628, and speaker 630. Other examples may include additional or fewer input and peripheral devices. The input and peripheral devices 622 are connected to the at least one processing device 602 through an input/output interface 624 that is coupled to the bus 606. In accordance with the examples described above, the microphone 626 may include microphone 222 and microphone 626 can be used by the computing device 202 to detect whether a subject is talking to reduce false alarms when measuring respiration rate. The LEDs 628 and speaker 630 can be part of the alarm 220 (see FIG. 2) that generates a visual or audible alert when an abnormal respiration rate is measured.

The computing device 202 further includes a communication device 632 configured to establish communication across a network such as the data communication network 110 (see FIG. 1). In some embodiments, the computing device 202 is connected to the network through a network interface, such as a wireless network interface 634.

In yet other embodiments, the communication device 632 is capable of short-range wireless communication. Short-range wireless communication is one-way or two-way shortrange to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The computing device 202 typically includes at least some form of computer-readable media. Computer readable media includes any available media that can be accessed by the computing device 202. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, or any other medium that can be used to store the desired information and that can be accessed by the computing device 202. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wireless media such as acoustic, radio frequency, infrared, and other wireless media. Combinations of any of the above are also included within the scope of computer readable media.

The computing device 202 is an example of programmable electronics, which may include one or more computing devices, and when multiple computing devices are included, such computing devices can be coupled together with a suitable data communication network so as to collectively perform the various functions, methods, or operations disclosed herein.

The computing device 202 can include a location identification device 636. The location identification device 636 is configured to identify the location or geolocation of the computing device 202. The location identification device 636 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

Embodiments of the present invention may be utilized in various distributed computing environments where tasks are performed by remote processing devices that are linked through a communications network in a distributed computing environment. The block diagrams depicted herein are just examples. There may be many variations to these diagrams described therein without departing from the spirit of the disclosure. For instance, components may be added, deleted, or modified.

The systems and method described herein result in a significant technical advantage. For example, the monitoring device 200 more efficiently captures vital sign measurements, including respiration rate, quality and depth of respiration, and the uniformity of breath, heart rate, ambulation index, agitation index, temperature, ambient noise in an environment of the subject S and/or blood pressure by automatically adjusting the sampling rate of the monitoring device based on a detection of abnormal vital sign value. Also, the monitoring device 200 facilitates remote monitoring of the patient which increases efficiency in nursing, decrease in ICU transfers, decrease in length of hospital stay and increase in patient satisfaction. Further, the monitoring device 200 may improve the quality of patient care or patient experience by facilitating direct communication between the patient and the healthcare provider and improve workflows within the healthcare facility by providing additional context for consumption by the healthcare provider prior to entering the patient room or communicating with the patient.

The description and illustration of the various examples provided in this application are not intended to limit or restrict the scope of the invention as claimed in any way. The examples provided in this application are considered sufficient to convey possession and enable others to make and use the best mode of the claimed invention. The claimed invention should not be construed as being limited to any embodiment, example, or detail provided in this application. Regardless, whether shown and described in combination or separately, the various features (both structural and methodological) are intended to be selectively included or omitted to produce an embodiment with a particular set of features.

Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A device for monitoring a subject, the device comprising:
one or more sensors; and
a computing device including:
one or more processors; and
memory storing instructions, which when executed by the one or more processors cause the one or more processors to:
use the one or more sensors to measure one or more vital sign values of the subject at a predetermined frequency;
receive a communication message from the subject;
tag the communication message with a timestamp; and
transmit the communication message and the one or more vital sign values measured proximate the timestamp to a computing device associated with a healthcare provider.

2. The device of claim 1, wherein the instructions when executed by the one or more processors further cause the one or more processors to:
receive a second communication message from the computing device associated with the healthcare provider, wherein the second communication message is in response to the communication message from the subject; and
present the second communication message to the subject.

3. The device of claim 2, wherein the communication message and the second communication message are audio messages.

4. The device of claim 1, further comprising one or more of a battery, an antenna, a microphone, a speaker, and a timer.

5. The device of claim 4, wherein presenting the second communication message includes interfacing with the speaker and causing playback of the second communication message.

6. The device of claim 1, wherein the one or more sensors include one or more of a respiration rate monitor, a heart rate monitor, a motion detector, a thermometer, and a blood pressure monitor.

7. The device of claim 1, wherein the one or more vital sign values of the subject include at least one of a respiration rate, a quality of respiration, a depth of respiration, a uniformity of breath, a heart rate, an irregularity of heartbeat ambulation index, an agitation index, a body temperature, an ambient noise level of an environment of the subject, or a blood pressure.

8. The device of claim 1, wherein the instructions when executed by the one or more processors further cause the one or more processors to:
determine whether at least one vital sign value of the one or more vital sign values is outside an expected range for the at least one vital sign value; and
upon determining that the at least one vital sign value is outside the expected range for the at least one vital sign, change the predetermined frequency at which the at least one vital sign value is measured.

9. The device of claim 8, wherein changing the predetermined frequency at which the at least one vital sign value is measured includes increasing the predetermined frequency.

10. The device of claim 1, wherein the instructions when executed by the one or more processors further cause the one or more processors to:
determine whether the communication message includes a preset keyword;
upon determining that the communication message includes the preset keyword, transmit the communication message to a voice assistant application;
receive a third communication message from the voice assistant application responding to the communication message, wherein the third communication message is an audio message; and
cause playback of the third communication message.

11. A method of monitoring a subject, the method comprising:
measuring one or more vital sign values of the subject at a predetermined frequency;
receiving a communication message from the subject;
tagging the communication message with a timestamp; and
transmitting the communication message and the one or more vital sign values measured proximate the timestamp to a computing device associated with a healthcare provider.

12. The method of claim 11, further comprising:
receiving a second communication message from the computing device associated with the healthcare provider, wherein the second communication message is in response to the communication message from the subject; and
presenting the second communication message to the subject using a monitoring device.

13. The method of claim 12, wherein the communication message and the second communication message are audio messages.

14. The method of claim 13, wherein presenting the second communication message includes interfacing with a speaker associated with the monitoring device and causing playback of the second communication message.

15. The method of claim 11, wherein the monitoring device is a wearable device that includes a microphone and a speaker.

16. The method of claim 11, wherein the one or more vital sign values of the subject include at least one of a respiration rate, a quality of respiration, a depth of respiration, a uniformity of breath, a heart rate, an irregularity of heartbeat ambulation index, an agitation index, a body temperature, an ambient noise level, and a blood pressure.

17. The method of claim 11 further comprising:
determining whether at least one vital sign value of the one or more vital sign values is outside an expected range for the at least one vital sign value; and
upon determining that the at least one vital sign value is outside the expected range, changing the predetermined frequency at which the at least one vital sign value is measured.

18. The method of claim 17, wherein changing the predetermined frequency at which the at least one vital sign value is measured includes increasing the predetermined frequency.

19. The method of claim 11, further comprising:
determining whether the communication message includes a preset keyword;
upon determining that the communication message includes the preset keyword, transmitting the communication message to a voice assistant application;
receiving a third communication message from the voice assistant application responding to the communication message, wherein the third communication message is an audio message; and
causing playback of the third communication message.

20. A method of communicating with a healthcare provider using a monitoring device, the method comprising:
measuring one or more vital sign values of a subject at regular time intervals;
upon measuring the one or more vital sign values, transmitting the one or more vital sign values to a computing device associated with the healthcare provider for display;
receiving a first communication message from the subject, wherein the first communication message is an audio message;
determining whether the first communication message includes a preset keyword;
upon determining that the first communication message does not include the preset keyword, tagging the first communication message with a first timestamp;
transmitting the first communication message with the first timestamp and the one or more vital sign values measured proximate the timestamp to a computing device associated with the healthcare provider;
receiving, at the monitoring device a second communication message from the computing device associated with the healthcare provider, wherein the second communication message includes an audio message responding to the first communication message; and
interfacing with a speaker associated with the monitoring device and causing playback of the second communication message on the speaker.
